(19) United States
(12) Patent Application Publication
(10) Pub. No.: US 2018/0021563 A1
(43) Pub. Date: Jan. 25, 2018

(54) IONTOPHORETIC DEVICE, ARRANGEMENT AND METHOD

(71) Applicant: KONINKLIJKE PHILIPS N.V., EINDHOVEN (NL)

(72) Inventors: Anja van de Stolpe, Vught (NL); Mark Thomas Johnson, Eindhoven (NL); Alwin Rogier Martijn Verschueren, Eindhoven (NL); Freek van Hemert, Dordrecht (NL)

(21) Appl. No.: 15/550,090

(22) PCT Filed: Feb. 25, 2016
(86) PCT No.: PCT/EP2016/053901

§ 371 Date: Aug. 10, 2017

(30) Foreign Application Priority Data
Mar. 9, 2015 (EP) 15158225.1

(51) Int. Cl. A61N 1/04; A61N 1/30

(52) U.S. Cl. CPC A61N 1/0428; A61N 1/30; A61N 1/303; A61N 1/0496

(57) ABSTRACT

Disclosed is an iontophoretic device (100) for applying a cutaneous DC electrical field, the device comprising a first skin contact electrode (120) comprising a free sodium ion reservoir (122) separated from the skin (300) by a first ion-permeable barrier (124); and a second skin contact electrode (130) spatially separated from the first skin contact electrode, the second skin contact electrode comprising a free chloride ion reservoir (132) separated from the skin by a second ion-permeable barrier (134). Also disclosed is an arrangement including such a device and a method of operating such a device.

### FIELD OF THE INVENTION

The invention relates to an iontophoretic device for applying a cutaneous (epidermis and dermis) DC electrical field to the skin of a subject and, to an arrangement including such a device and to a method of operating such a device.

### BACKGROUND OF THE INVENTION

It is well-known that electrical fields play several roles in biological processes, i.e. physiology, such as embryonic development, tissue recycling and repair, e.g. in hair, skin and intestinal wall, ion transport like in the kidney and intestines, and pathophysiology, e.g. cancer, wound healing and regenerative medicine. All types of cells are in principle electrically active: in a cell type-dependent manner they generate either constant, e.g. most epithelial cell types (around 40 mV over the whole cell layer), or alternating, e.g. cardiac cell, nerve cells and many other cell types for cell signaling, voltage differences in the order of 100-200 mV over the outer cell phospholipid bilayer membrane, mitochondrial and nuclear membranes wherein the inner side of the membrane is negatively charged.

These insights have led to applications in which alternating electrical fields are applied to cells in order to influence physiological processes in the human or animal body, e.g. cell division processes. For example, US 2012/0283726 A1 discloses an apparatus for destroying or inhibiting the growth of rapidly dividing, e.g. tumor, cells. The apparatus comprises an Alternating Current (AC) voltage source and a plurality of insulated electrodes connected to the AC voltage source for placement against the patient's body. The AC voltage source and the electrodes are configured such that a first AC field having a first frequency and a second AC field having a different second frequency are imposed in the target region of the patient, wherein the AC fields have frequency characteristics corresponding to a vulnerability in the rapidly dividing cells, such that the cells are damaged in the late anaphase or telophase stages of cell division by application of strong enough AC fields whilst leaving non-dividing cells substantially unaffected.

A wound-healing application is disclosed in WO 2014/145239 A1, which discloses a non-user controllable electro-therapy device including a microprocessor generating a non-user controllable frequency dependent mixed AC electrical signal through one or more electrodes, wherein the mixed electrical signal is a combination of at least two different frequencies, a first frequency having a first minimum and maximum micro-ampere range and a second frequency having a different second minimum and maximum micro-ampere range. The higher of the two frequencies is superimposed on the lower frequency, creating a current intensity window as an envelope along a profile of the lower frequency. The mixed AC electrical signal is automatically applied for a pre-determined period of time, and amplitude and/or duration and/or frequencies is varied according to a pre-set schedule programmed into a controller coupled to the one or more electrodes.

### SUMMARY OF THE INVENTION

The application of alternating currents to cellular material is not without controversy, as there are concerns that such alternating electrical fields may cause damage to healthy cellular material, especially when field strengths and/or frequencies in excess of normal physiological field strengths are applied. Moreover, the generation of an alternating current requires dedicated hardware components, which add to the cost and complexity of the field-generating devices, which can be particularly undesirable when the device is to be disposable.

It is an objective of the invention to overcome difficulties associated with AC operated devices.

This objective is at least partly reached with the current invention.

The invention is defined by the independent claims. The dependent claims define advantageous embodiments.

The invention thus provides a device for influencing stem cell division using direct current (DC) electrical fields having physiological field strengths, an arrangement including such a device and a method of operating such a device.

The invention is based on the discovery by the present inventors that stem cells will exhibit asymmetric cell differentiation when subjected to a DC electric field of physiological field strength during cell division, associated with asymmetric distribution of protein receptors in the cell membrane together with alignment of the cell division spindle in the line of the electrical field, wherein one of the daughter cells remains a stem cell whereas the other of the daughter cells will differentiate (vide infra) due to loss of membrane-associated protein receptors which are needed to retain stemness. By limiting the number of stem cells in the stem cell pool, the ability to replenish lost mature differentiated cells is limited, thereby significantly reducing the ability of a particular feature to grow from stem cell differentiation, as the differentiated daughter cells are typically incapable or less capable of division, or at least are not capable of unlimited cell division. This can be exploited to suppress regeneration of a hair follicle or for example growth of a tumor/cancer.

Mi Zhao et al., in Proc. Natl. Acad. Sci. USA, 1999, 96(9), pages 4942-4946, report that dividing differentiated cells, i.e. corneal epithelial cells, align in the direction of the electrical field. While in non-dividing cells transforming growth factor β receptor type II distributes towards the cathode, during cell division the receptor accumulated symmetrically at both poles at the cell cleavage site and daughter cells, suggesting that the TGF-beta receptor does not distribute asymmetrically over daughter cells, at least not in non-stem cells, when applying a physiological DC electric field across the dividing cells.

The inventors have further realized that a device may be provided for invoking such asymmetric stem cell division across a skin area of a subject such as for example a human or animal patient, for treatment purposes, for instance to deplete cancerous stem cells, e.g. carcinomas or benign tumor stem cells, thereby halting the growth of such cancers or tumors, or for cosmetic purposes, for instance to deplete the stem cell niche in hair follicles to reduce unwanted hair growth in areas of the skin. Such a device will need to be applied to an area of skin of the subject for a period of time that is long enough to effect the desired asymmetric stem division-associated daughter cell differentiation, e.g. at least 1 hour, and more preferably 6-10 hours, e.g. 8 hours, such as during sleep, to affect more cell divisions. During this period, a DC electric field will be applied to the area of skin between the electrodes of the device. As the skin contains a saline-like solution containing predominantly Na⁺ and Cl⁻ ions, the applied DC electric field will cause the migration of these Na⁺ and Cl⁻ ions to the cathode and anode respectively. In order to replenish these ions and maintain the electrolyte balance of the area of skin under treatment, the device of the present invention contains a first electrode (acting as an anode) comprising a free sodium ion reservoir separated from the skin by a first ion-permeable barrier and a second skin contact electrode (acting as a cathode) spatially separated from the first skin contact electrode, the second skin contact electrode comprising a free chloride ion reservoir separated from the skin by a second ion-permeable barrier to replenish the migrating ions in the skin. The ion-permeable barriers ensure that the skin is not in direct contact with the media containing the free sodium (Na⁺) and chloride (Cl⁻) ions respectively, thus preventing damage to the skin, e.g. from burning in case the media are strongly alkaline or acidic.

The device preferably is for providing a cutaneous (epidermis and dermis) DC field to the skin.

The device may be made wearable. This can mean that it is suitable for remaining attached to the skin of the subject for prolonged periods of time. Furthermore, wearable can mean that it remains attached to the skin of the subject during periods in which that subject can perform normal activities of daily life.

The first skin contact electrode and the second skin contact electrode may be integrated in a skin patch (preferably one that is adhesive to the skin) to facilitate application of the device to the area of the skin to be treated. The patch can define a fixed and predetermined distance between edges of the first and second skin electrodes. Alternatively, Skin electrodes can be made moveably attached to the patch to allow user defined distances between them for accommodating different areas of skin to be provided with the DC field.

Preferably, the free sodium ion reservoir contains at least 1 mmol of sodium ions; and the free chloride ion reservoir contains at least 1 mmol of chloride ions. This facilitates the use of the device under the application of physiological DC electric fields for about 8 hours at least, as the amount of free Na⁺ and Cl⁻ ions is sufficient to replenish migrating Na⁺ and Cl⁻ ions in the skin as induced by the applied physiological DC electric field over that period of time.

In an embodiment, the first ion-permeable barrier and the second ion-permeable barrier comprise respective salt bridges. This has the advantage that the barriers have a low intrinsic resistivity, thereby facilitating the application of the DC electric field across the area of skin to be treated.

Each salt bridge may comprise a gel including an isotonic NaCl concentration to minimize the risk of skin irritation by the contact between the skin and the salt bridge.

Alternatively, the first ion-permeable barrier and the second ion-permeable barrier comprise respective ion-exchange membranes in order to facilitate the migration of the free Na⁺ and Cl⁻ ions from the respective reservoirs to the skin.

In an embodiment, the free sodium ion reservoir comprises an electrolyte solution including free sodium ions; and the free chloride ion reservoir comprises an electrolyte solution including free chloride ions. This has the advantage that a large amount of free Na⁺ and Cl⁻ ions can be provided, thus facilitating the prolonged use of the device.

The respective electrolyte solutions may be buffered solutions to reduce the harmfulness of the electrolyte solutions upon unexpected exposure of the skin to the solutions.

Alternatively, the sodium ion reservoir may comprise a hydrogel including free sodium ions; and the chloride ion reservoir comprises a hydrogel including free chloride ions to provide a relatively harmless source of such free ions.

In a preferred embodiment, the wearable iontophoretic device further comprises an integrated DC supply source such as a battery having a first supply, terminal conductively coupled to the first skin contact electrode, and a second supply terminal conductively coupled to the second skin contact electrode. This provides a self-contained wearable iontophoretic system that does not require connecting to a separate power supply, thus yielding a wearable iontophoretic device that is particularly easy to use.

Alternatively, and in accordance with another aspect of the present invention, an arrangement including the wearable iontophoretic device is provided in which the arrangement further comprises a DC supply source separate to the wearable iontophoretic device for providing a direct voltage to the wearable iontophoretic device over a defined period of time, said DC supply source comprising a first supply terminal for conductively connecting to the first skin contact electrode and a second supply terminal for conductively connecting to the second skin contact electrode. This has the advantage that the disposable wearable iontophoretic device does not require an integrated DC power supply, thus reducing the cost of this disposable device, which comes at the expense of requiring more user involvement as the user has to connect the DC power supply to the wearable iontophoretic device prior to use.

The DC supply source may be adaptable to generate a cutaneous DC electric field in the range of 0.1-10 V/cm, or preferably 0.5-2 V/cm, such as about 1 V/cm. These are typical physiological DC electric fields that can induce the desired asymmetric stem cell differentiation.

According to yet another aspect, there is provided a method of operating the wearable iontophoretic device of any of the aforementioned embodiments, the method comprising bringing the wearable device into contact with an area of skin such that the first skin contact electrode and the second skin contact electrode contact said area; and generating a cutaneous DC electrical field across said area for a period of time by providing the first terminal and the second terminal with a potential difference for said period of time in order to induce asymmetric stem cell division in said area. This method may therefore be used to deplete the stem cell niche in the skin area subjected to the cutaneous DC electrical field whilst maintaining electrolyte balance in the skin area.

In an embodiment, said area comprises hair follicles, and said cutaneous DC electrical field is applied for a period of time sufficient to induce asymmetric stem cell division in said hair follicles. This equates to a cosmetic treatment of the skin area by reducing or suppressing hair growth in this area.

Preferably, said period of time is at least 1 hour to induce the aforementioned asymmetric differentiation in a sufficient number of stem cells in the skin area under treatment.

### BRIEF DESCRIPTION OF THE DRAWINGS

Embodiments of the invention are described in more detail and by way of non-limiting examples with reference to the accompanying drawings, wherein:

FIG. 1 schematically depicts an experimental set-up for demonstrating proof of concept of the present invention;

FIG. 2 shows how the axis of the cell division spindle aligns with the applied DC electrical field;

FIG. 3 is a microscope image of a MDA-MB-231 cell after application of the DC electrical field, prior to initiation of cell division;

FIG. 4 is a microscope image of a MDA-MB-231 cell after application of the DC electrical field, during cell division;

FIG. 5 is a microscope image of two MDA-MB-231 daughter cells resulting from a cell division during application of the DC electrical field;

FIG. 6 schematically depicts a wearable iontophoretic device according to an embodiment;

FIG. 7 schematically depicts an arrangement according to an embodiment including the wearable iontophoretic device of FIG. 6;

FIG. 8 schematically depicts a wearable iontophoretic device according to another embodiment;

FIG. 9 schematically depicts the application of a wearable iontophoretic device according to an embodiment to a skin area with hair follicles;

FIG. 10 schematically depicts the application of a wearable iontophoretic device according to an embodiment to a skin area with a growth anomaly such as a tumor; and

FIG. 11 is a flow chart of a method of operating a wearable iontophoretic device according to an embodiment.

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

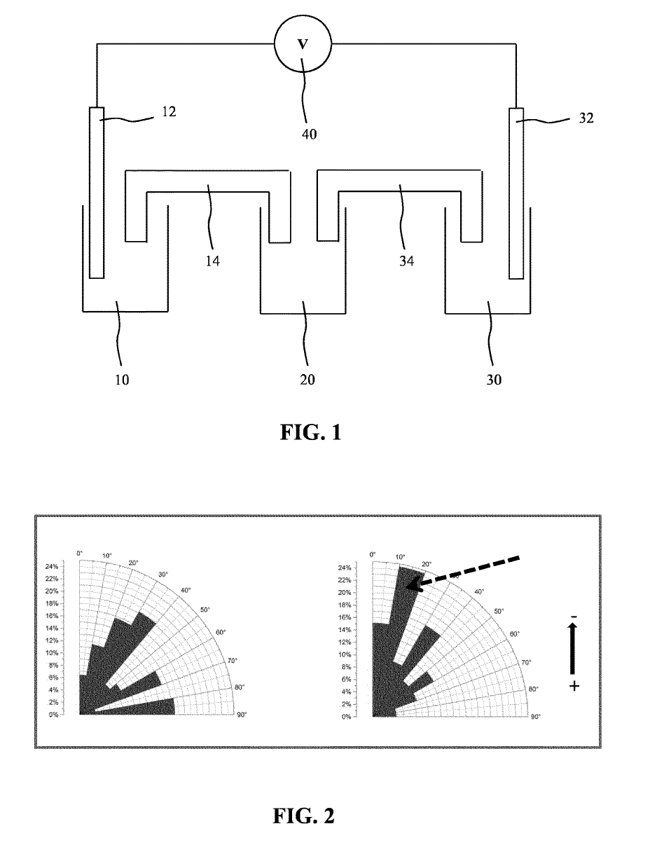

In order to demonstrate proof of concept, the inventors have performed an experiment using the experimental set-up schematically depicted in FIG. 1. In the set-up of FIG. 1, a culture of MDA-MB-231 breast cancer epithelial cells, cultured/passaged according to ATCC provided protocol, were cultured in a reservoir 20 of a microfluidic chip containing the same culture medium, which reservoir 20 was in fluid connection with an electrolyte reservoir 10 containing a 150 mM NaOH solution in water via an agarose saline salt bridge 14, and with an electrolyte reservoir 30 containing a 150 mM HCl solution in water via an agarose saline salt bridge 34. Salt bridges 14, 34 prevent the culture medium from contamination by contaminants from the electrolyte reservoirs 10, 30. MDA-MB-231 breast cancer epithelial cells (from the ATCC, USA) were used because such cancer cells exhibit many stem cell-like characteristics.

A Pt-electrode 12 configured as anode was inserted into the electrolyte reservoir 10 and a Pt-electrode 32 configured as cathode was inserted into the electrolyte reservoir 30 and connected to a Keithley 2410 source meter acting as a DC power supply 40 providing a 13.5 V potential across the set-up, which was measured with a Keithley 6517 electrometer to produce a DC electric field of between 1 and 6 V/cm across the reservoir 20. Cells were cultured in the culture chamber 20 in the microfluidic device (FIG. 1). After incubating the cells first with Nocadazole for 12.5 hours to synchronize MDA-MB-231 cell division, a DC electrical field (6 V/cm) was applied during 3.5 hours in the presence of an electrical field followed by an additional 2.5 hours after removal of Nocadazole. The results of the applied electrical field on the alignment of the MDA-MB-231 cells are shown in FIG. 2. The left pane shows the spindle angle distribution of 221 cells in the absence of an applied DC electrical field and the left pane shows the distribution of the spindle angle distribution relative to the applied DC electrical field of 239 cells in a DC electrical field of 1 V/cm applied for 2 hours. As highlighted by the arrow, the cells subjected to the DC electrical field demonstrate a strong alignment of the cell division spindles with the direction of the applied electrical field, with cell division cleavage plane oriented perpendicular to the field direction.

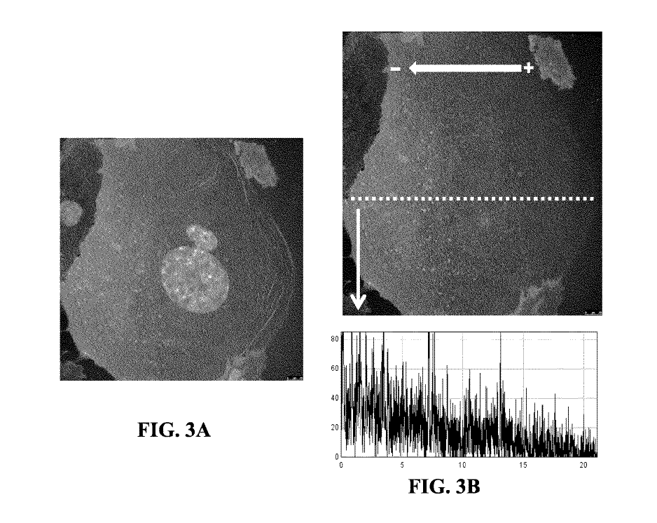

Cells were subsequently fixated and immunofluorescently stained with an antibody against DAPI (blue), alpha tubulin (green) and the EGF receptor (red) (FIG. 3). EGFR staining distribution over the cells was measured and quantified using an intensity-based algorithm along a line drawn through the middle of the cell, in the direction of the applied electrical field (FIG. 3-5).

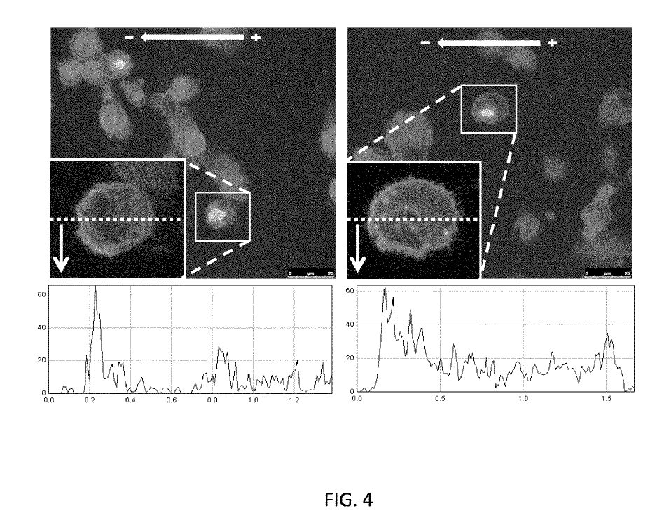

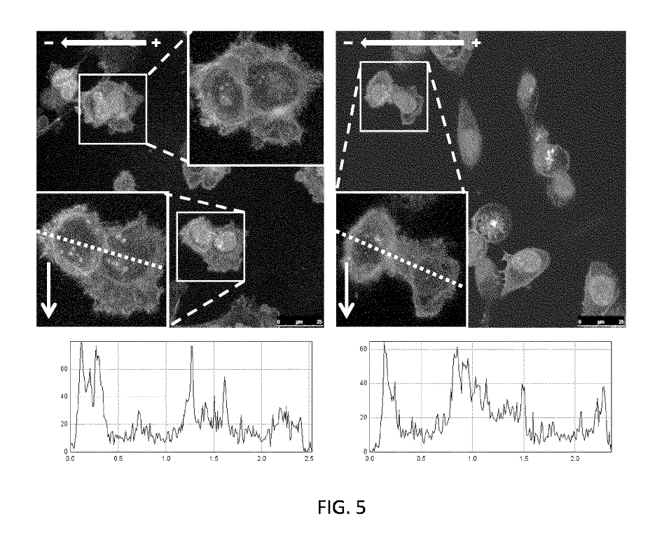

Prior to actual cell division an asymmetric distribution of membrane EGF receptors was observed in the direction of the cathode (FIGS. 3A and 3B). During cell division (M-phase) an asymmetric distribution of membrane EGF receptors was still observed in the direction of the cathode (FIG. 4). The cell division spindle has been stained according to standard protocol, and has aligned to the DC electrical field (FIG. 4). After cell division an asymmetric distribution of membrane EGF receptors was observed over the two daughter cells, with the cell ending up closest to the cathode containing most of the EGF receptors (FIG. 5).

Hence, these results clearly demonstrate that proteins such as the EGF receptor in the cell membrane accumulate preferentially in one of the two daughter cells to be formed after cell division, i.e. in the cell adjacent the cell division spindle that is proximal to the cathode, thus clearly indicating that the proximal cell will remain a stem cell whereas the cell distal to the cathode (i.e. proximal to the anode) will differentiate at least due to the absence of the proteins required for imparting the stem cell characteristics on the distal cell.

FIG. 6 schematically depicts a wearable iontophoretic device 100 according to an embodiment of the present invention. The device 100 preferably is a disposable device for applying to an area of skin to be treated, as will be explained in more detail below. The device 100 typically comprises a carrier medium 110 in which the first electrode 120 and the second electrode 130 are mounted or embedded. The first electrode 120 is spatially separated from the second electrode 130 with the space between the first electrode 120 and the second electrode 130 corresponding to the area of skin to be treated. The carrier medium 110 may be used to place the first electrode 120 and the second electrode 130 against the area of skin to be treated. The carrier medium 110 may be a non-adhesive medium such as a wearable item, e.g. a bracelet, a strap, and so on, that can be applied to the area of skin to be treated. Alternatively, the carrier medium 110 may be an adhesive patch or the like including the first electrode 120 and the second electrode 130, which has the advantage that the carrier medium 110 may be applied to parts of the skin to which it may be difficult to apply a non-adhesive medium. Any suitable type of adhesive patch may be used as a carrier medium 110; as adhesive patches are well-known per se, this will not be explained in further detail for the sake of brevity.

The first electrode 120 and the second electrode 130 may be made of any suitable electrically conductive material and are preferably made of a metal or metal alloy such as a platinum electrode, a platinum-coated titanium electrode, a silver electrode, and so on. Other suitable types of electrodes will be immediately apparent to the skilled person. The first electrode 120 and the second electrode 130 may be made of the same material or may be made of different materials. The first electrode 120 may comprise a first terminal 126 for connecting the first electrode 120 and the second electrode 130 may comprise a second terminal 136 for connecting the second electrode 130 to a DC power supply as will be explained in more detail below. The first terminal 126 and the second terminal 136 may have any suitable shape and may be made of any suitable material. In an embodiment, the first terminal 126 is integral to the first electrode 120 and the second terminal 136 is integral to the second electrode 130.

The first electrode 120 and the second electrode 130 may have any suitable shape. In an embodiment, the first electrode 120 and the second electrode 130 are shaped to surround or enclose the area of skin to be treated, e.g. may have a hemispherical shape. The device 100 may comprise a plurality of first electrodes 120 and a plurality of second electrodes 130, e.g. an array of first electrodes 120 spatially separated, e.g. opposing, an array of second electrodes 130 which for instance may be advantageous when treating a relatively large area of skin. Other suitable spatial arrangements of the first electrode 120 and the second electrode 130 will be immediately apparent to the skilled person.

The first electrode 120 is typically configured to act as the anode of the device 100. To this end, the first electrode 120 further comprises a first reservoir 122 for delivering Na⁺ ions to the area of skin in contact with the first electrode 120. The first reservoir 122 is separated from the skin by a first barrier 124 which prevents direct contact between the contents of the first reservoir 122 and the skin, but allows ions including Na⁺ ions to travel from the first reservoir 122 to the skin and vice versa.

The second electrode 130 is typically configured to act as the cathode of the device 100. To this end, the second electrode 130 further comprises a second reservoir 132 for delivering Cl⁻ ions to the area of skin in contact with the second electrode 130. The second reservoir 132 is separated from the skin by a second barrier 134 which prevents direct contact between the contents of the second reservoir 132 and the skin, but allows ions including Cl⁻ ions to travel from the second reservoir 132 to the skin and vice versa.

In operation, first electrode 120 and the second electrode 130 of the device 100 will be connected to a DC power supply such that a cutaneous DC electric field is created across the area of skin between the first electrode 120 and the second electrode 130. Such a cutaneous DC electric field preferably has a field strength in an endogenous physiological range, e.g. in the range from about 0.1-10 V/cm, preferably in the range from 0.5-2 V/cm, such as about 1 V/cm. As explained above, it has been demonstrated that under such field strengths dividing stem cells align with the applied electric field and exhibit asymmetric cell division. Consequently, the application of the cutaneous DC electric field can be used to induce asymmetric cell division of the stem cells in the area of skin subjected to this cutaneous DC electric field, i.e. the area of skin in between the first electrode 120 and the second electrode 130. Because the process of cell division is a stochastic process of the timescale of hours, the device 100 should be applied to the area of skin to be treated for at least one hour and preferably for several hours, e.g. up to 10 hours or more, in order to substantially deplete the stem cell pool in the area of skin under treatment. For example, the device 100 may be applied at night time when the patient is sleeping, which has the additional advantage it provides a patient with privacy, which may be desirable if the device 100 is applied to a visible area of skin, such as the upper lip for example in case of a hair growth suppressing treatment as will be explained in more detail below.

As the skin contains a saline-like solution containing predominantly Na⁺ and Cl⁻ ions, the applied DC electric field will cause the migration of these Na⁺ and Cl⁻ ions to the cathode and anode respectively. In order to replenish these ions and maintain the electrolyte balance of the area of skin under treatment, the device of the present invention contains a first electrode (acting as an anode) comprising a free sodium ion reservoir separated from the skin by a first ion-permeable barrier and a second skin contact electrode (acting as a cathode) spatially separated from the first skin contact electrode, the second skin contact electrode comprising a free chloride ion reservoir separated from the skin by a second ion-permeable barrier to replenish the migrating ions in the skin. In addition to Na⁺, the anode reservoir may also contain K⁺, Ca²⁺ and Mg²⁺ for example in the molar ratio Na⁺:K⁺:Ca²⁺:Mg²⁺=140:4:2:1 to mimic the composition of cations in interstitial fluid. And likewise, in addition to Cl⁻, the athode reservoir may also contain HCO₃⁻, H₂PO₄⁻ and SO₄²⁻, for example in molar ratio C⁻:HCO₃^{-:}H₂PO₄⁻:SO₄²⁻=122:25:1:1 to mimic the composition of anions in interstitial fluid.

The following half reactions will occur at the surface of the first electrode 120 and the second electrode 130:

H₂O (l)→2H⁺ (aq)+½O₂(g)+2e⁻ Anode:

2H₂O (l)+2e−→H₂ (g)+2 OH⁻(aq) Cathode:

The reservoirs 122, 132 may be involved in recombination reactions with the species generated at the first electrode 120 (anode) and second electrode 130 (cathode) respectively:

2NaOH (aq)+2H⁺ (aq)→2H₂O (l)+2Na⁺ (aq) Anode recombination:

2HCl (aq)+2OH⁻(aq)→2H₂O (l)+2Cl⁻(aq) Cathode recombination:

As previously explained, the first reservoir 120 and the second reservoir 130 are involved in replenishing ions in the skin that migrate towards the anode and cathode as a result of the applied DC electric field and may be involved in the above recombination reactions. In an embodiment, the first reservoir 122 comprises an electrolyte solution including Na⁺ ions, such as a NaOH solution. Preferably, this first reservoir is alkaline (containing OH⁻ ions) which would recombine or neutralize H⁺ ions formed by the water electrolysis half reaction at the anode. In order to protect the skin from damaging direct exposure to such a caustic electrolyte solution, the first reservoir 122 is separated from direct contact with the skin by a first barrier 124, which is ion-permeable to allow transport of ions between the first reservoir 122 and the skin in contact with the first barrier 124. A non-limiting example of a suitable first barrier 124 is an agarose gel salt bridge comprising an isotonic saline solution (about 150 mM) although other types of salt bridge or other suitable ion-exchange barriers that facilitate such ion exchange without directly exposing the skin to the contents of the first reservoir 122 are equally feasible, e.g. ion-permeable membranes such as ion exchange polymer membranes. In addition to Na⁺, the anode salt bridge may also contain K⁺, Ca²⁺ and Mg²⁺ for example in the molar ratio Na⁺:K⁺:Ca²⁺:Mg²⁺=140:4:2:1 to mimic the composition of cations in interstitial fluid. And likewise, in addition to Cl⁻, the cathode salt bridge may also contain HCO₃⁻, H₂PO₄⁻ and SO₄²⁻ for example in molar ratio Cl⁻:HCO₃^{-:}H₂PO₄⁻:SO₄²⁻=122:25:1:1 to mimic the composition of anions in interstitial fluid.

As an alternative to a NaOH solution, the first reservoir 122 may contain a Na⁺-based buffer solution, such as a 1M NaHCO₃ buffer, which has a pH of about 8 and as such is less harmful to the skin in case of direct contact with the buffer solution. It should however be understood that the first reservoir 122 is not limited to electrolyte solutions to provide the free Na⁺ ions for migration to the skin. In an alternative embodiment, the first reservoir 122 contains a hydrogel, e.g. a sodium polyacrylate-based hydrogel, a sodium pentaborate pentahydrate hydrogel, and so on.

The first reservoir 122 preferably comprises at least 1 mmol of free Na⁺ ions as this is typically the amount of Na⁺ ions in the skin that migrate towards the cathode, i.e. the second electrode 130 during application of the DC electric field over a period of time of about 8 hours, e.g. during a night's sleep of the patient, such that at least 1 mmol of free Na⁺ ions in the first reservoir 122 ensures that the displaced Na⁺ ions in the skin can be adequately replenished. The first reservoir 122 preferably contains a negligible amount of Cl⁻ ions and more preferably contains no Cl⁻ ions to avoid the generation of (noticeable amounts of) Cl₂ gas at the anode, which causes an unpleasant smell that may deter a patient from using the device 100.

In an embodiment, the second reservoir 132 comprises an electrolyte solution including Cl⁻ ions, such as a HCl solution. Preferably, this second reservoir is acidic (containing H⁺ ions) which would recombine or neutralize OH⁻ ions formed by the water electrolysis half reaction at the cathode. In order to protect the skin from damaging direct exposure to such a caustic electrolyte solution, the second reservoir 132 is separated from direct contact with the skin by a second barrier 134, which is ion-permeable to allow transport of ions between the second reservoir 132 and the skin in contact with the second barrier 134. A non-limiting example of a suitable second barrier 134 is an agarose gel salt bridge comprising an isotonic saline solution (about 150 mM) although other types of salt bridge or other suitable ion-exchange barriers that facilitate such ion exchange without directly exposing the skin to the contents of the second reservoir 132 are equally feasible, e.g. ion-permeable membranes such as ion exchange polymer membranes.

As an alternative to a HCl solution, the second reservoir 132 may contain a Cl⁻-based buffer solution, such as a 1M NH₄Cl buffer, which has a pH of about 5 and as such is less harmful to the skin in case of direct contact with the buffer solution. It should however be understood that the second reservoir 132 is not limited to electrolyte solutions to provide the free Cl⁻ ions for migration to the skin. In an alternative embodiment, the second reservoir 132 contains a chloride releasing hydrogel, for example a poly dimethyldiallylammonium chloride based hydrogel.

The second reservoir 132 preferably comprises at least 1 mmol of free Cl⁻ ions as this is typically the amount of Cl⁻ ions in the skin that migrate towards the anode, i.e. the first electrode 120 during application of the DC electric field over a period of time of about 8 hours, e.g. during a night's sleep of the patient, such that at least 1 mmol of free Cl⁻ ions in the second reservoir 132 ensures that the displaced Cl⁻ ions in the skin can be adequately replenished.

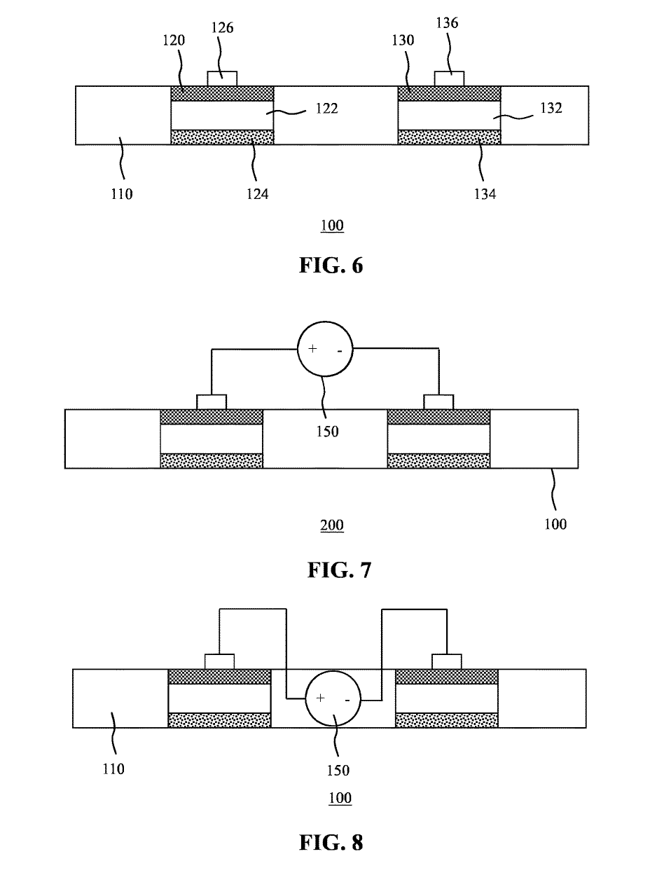

In an embodiment, the wearable iontophoretic device 100 may be connected to an external DC power supply 150 as schematically shown in FIG. 7 in order to provide the first electrode 120 and the second electrode 130 with the required potential difference to generate the cutaneous DC electric field with the desired field strength across the area of skin in between the first electrode 120 and the second electrode 130 when the device 100 is applied to the skin region to be treated by the device 100. This yields an arrangement 200 including the wearable iontophoretic device 100 and a DC power supply 150 external to the wearable iontophoretic device 100. Any suitable DC power supply 150 may be used for this purpose, such as a mains-powered DC power supply or a battery-powered DC power supply. The DC power supply 150 may be arranged to provide a fixed output voltage or current such that the device does not require configuring by the user or alternatively may be a configurable power supply where the output power may be configured by the user.

An advantage of this arrangement 200 is that the disposable wearable iontophoretic device 100, e.g. a disposable skin patch or the like, does not include the power supply 150, thereby reducing the cost of the disposable part of the arrangement 200, which reduces the overall cost of the treatment to be applied by using the arrangement 200. However, a drawback of this arrangement is that it requires the user to connect the first terminal 126 and the second terminal 136 to the correct polarity supply terminal of the DC power supply 150 to ensure that the first electrode 120 operates as the anode and the second electrode 130 operates as the cathode. As will be understood, reversing this polarity will at least in part prohibit the first reservoir 122 and the second reservoir 132 from replenishing the area of skin on the treatment with Na⁺ and Cl⁻ ions as the ions in the respective reservoirs are now attracted rather than repelled by the first electrode 120 and second electrode 130 respectively. This may be avoided by giving the first terminal 126 and the second terminal 136 different shapes, such that each terminal is shaped to mate with a supply terminal of the DC power supply 150 having a complementary, i.e. matching, shape, to avoid such undesirable polarity reversals.

Nevertheless, in order to avoid user error and increase user convenience by not having to connect the wearable iontophoretic device 100 to a separate power supply, it may be preferable to provide a wearable iontophoretic device 100 comprising an integrated DC power supply 150 such as a battery, as schematically depicted in FIG. 8. The integrated DC power supply 150 typically stores a charge that is sufficient to maintain the cutaneous DC electric field with the desired field strength for the duration of the treatment of the skin area, e.g. up to 10 hours or more. Although this increases the cost of the disposable wearable iontophoretic device 100, it also increases the ease of use of the device 100 and eliminates potential user error as the first terminal 126 and the second terminal 136 are permanently connected to the appropriate terminals of the integrated DC power supply 150. As will be readily understood by the skilled person, in this embodiment the device 100 will be automatically activated when the first electrode 120, i.e. the first barrier 124 and the second electrode 130, i.e. the second barrier 134, are brought into contact with the skin, as the skin provides the conductive medium that allows a current to flow between the first electrode 120 and the second electrode 130.

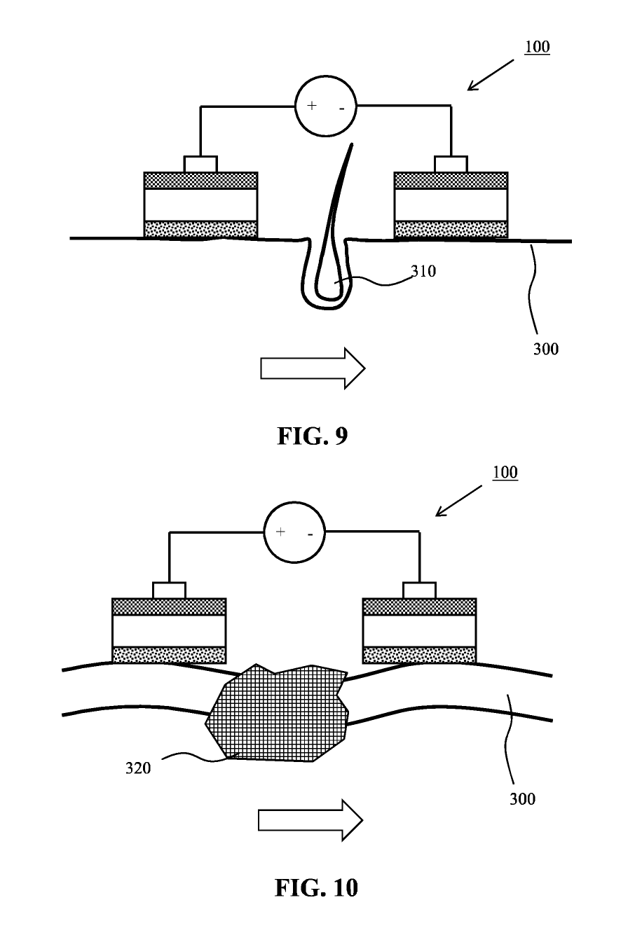

FIG. 9 schematically depicts a first example use case of the wearable iontophoretic device 100 (or arrangement 200) in which the device 100 is used to suppress hair growth in the area of skin under treatment. Excess hair growth in women is a clinical problem that is difficult to treat. For this reason, a safe and easy method for the removal of unwanted hair growth without shaving, waxing, treatment with hair removal creams or permanent removal of hair follicles using laser-induced necrosis is highly desirable, as it avoids the discomfort associated with such hair removal techniques. It has been previously reported by Snippert et al. in Cell, 2010, 143(1), pages 134-144, that stem cells in the dermal papilla divide symmetrically, such that based on the findings of the present inventors it can be expected that the induced asymmetric division of these stem cells by the application of the cutaneous DC electric field (as indicated by the block arrow) will prevent multiplication of stem cells in the hair follicles 310 in the area of skin between the first electrode 120 and the second electrode 130, thereby suppressing hair growth in this area.

The wearable iontophoretic device 100 may be used as a stand-alone treatment to suppress hair growth, where the user may daily apply the wearable iontophoretic device 100 over a period of time, e.g. two-four weeks at periodic intervals, e.g. once every 6-12 months, in order to effectively suppress hair growth in the area of the skin 300 under treatment. Alternatively, the wearable iontophoretic device 100 may be used in combination with temporary hair removal techniques, e.g. epilation, shaving, waxing or the like, in order to reduce the frequency at which such temporary hair removal techniques need to be employed in order to control unwanted hair growth in areas of the skin 300, e.g. on the upper lip of the patient.

Another example use case of the wearable iontophoretic device 100 (or arrangement 200) is schematically depicted in FIG. 10, in which the device 100 is used to suppress the growth of an anomaly 320 such as a benign or cancerous tumor, e.g. a melanoma, basal cell carcinoma or squamous cell carcinoma, in the area of skin 300 under treatment, here shown to reside in the upper layer (epidermis) of the skin 300 by way of non-limiting example. The device 100 is applied to the area of skin 300 including the anomaly 320 such that the first electrode 120 and second electrode 130 surround or contact the anomaly 320, thereby providing a DC electric field (as indicated by the block arrow) across the anomaly 320. It is well-documented that the growth of tumors is driven by continued symmetric stem cell division, as for instance disclosed by Snippert et al. in Cell. 2010 Oct. 1; 143(1): pages 134-4, thus constantly replenishing the pool of stem cells from which differentiated tumor cells can develop. Therefore, the periodic application of the wearable iontophoretic device 100 can be used to deplete the stem cell niche from which the tumor cells differentiate, and drive them towards differentiation, for instance to prevent cancer progression and in particular cancer metastasis in case of a cancerous tumor 320. Any suitable treatment frequency may be contemplated, such as the daily application of the wearable iontophoretic device 100 for a period of several hours, e.g. 6-10 hours, such as about 8 hours until complementary treatment to reduce the anomaly 320 has been successful, daily treatment for a period of 2-4 weeks 3-4 times a year, and so on.

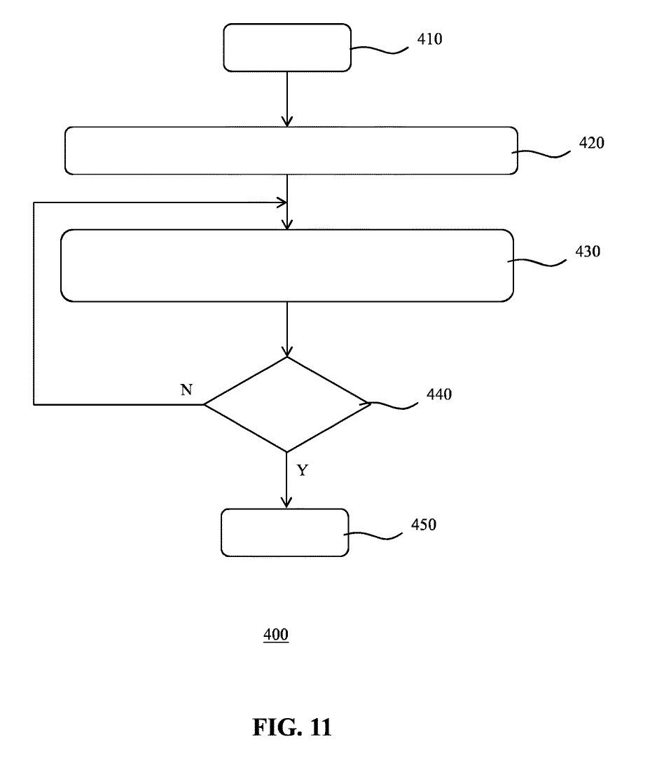

In each of these example use cases, the wearable iontophoretic device 100 may be operated in accordance with the method 400 as depicted by the flow chart in FIG. 11. The method 400 starts in step 410 by the provision of the wearable iontophoretic device 100, e.g.

a skin patch, bracelet, strap or the like as previously explained, after which the method 400 proceeds to step 420 in which the wearable iontophoretic device 100 is applied to the area of skin to be treated, e.g. an area comprising unwanted hair growth or comprising a tumorous anomaly as previously explained. This step may further comprise the removal of a non-adhesive protective film or layer in case of the wearable iontophoretic device 100 being an adhesive patch prior to the application of the wearable iontophoretic device 100 to the area of skin to be treated.

Next, the method 400 proceeds to step 430 in which the aforementioned cutaneous DC electric field is applied across the area of skin to be treated by the application of a potential difference between the first electrode 120 and the second electrode 130. This step may be automatically invoked by bringing the first electrode 120 and the second electrode 130 of the wearable iontophoretic device 100 into contact with the skin in case the first electrode 120 and the second electrode 130 are permanently coupled to a power supply 150 included in the device 100, as the skin in this embodiment provides the conductive medium through which a current between the first electrode 120 and the second electrode 130 can run as induced by this potential difference, as previously explained. Alternatively, this step may be invoked by connecting the first terminal 126 of the first electrode 120 and connecting the second terminal 136 of the second electrode 130 to an external DC supply source 150 and activating the external DC supply source 150 if necessary. The wearable iontophoretic device 100 preferably remains attached to the area of skin to be treated for at least 1 hour and more preferably for several hours, e.g. up to 8-10 hours or more, to ensure that a significant amount of stem cells in the area of skin to be treated is forced into asymmetric division by the applied DC electric field. This is symbolized by step 440, in which the wearable iontophoretic device 100 is maintained into contact with the area of skin to be treated until the treatment is completed and the method terminates in step 450, e.g. by the removal of the wearable iontophoretic device 100 from the area of skin under treatment.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

1. A iontophoretic device for applying a DC electrical field to a subject having a skin, the device comprising:
a first skin contact electrode comprising:
a free sodium ion reservoir containing at least 1 mmol of sodium ions; and
a first ion-permeable barrier,
the free sodium ion reservoir and the first ion-permeable barrier being arranged with respect to each other such that the first ion-permeable reservoir is at least partly between the free sodium ion reservoir and the skin of the subject when the first skin contact electrode is applied to the skin of the subject; and
the device further comprising:
a second skin contact electrode spatially separated from the first skin contact electrode, the second skin contact electrode comprising:
a free chloride ion reservoir containing at least 1 mmol of chloride ions; and
a second ion-permeable barrier;
the free chloride ion reservoir and the a second ion-permeable barrier being arranged with respect to each other such that the second ion-permeable barrier is at least partly between the free chloride ion reservoir and the skin of the subject when the second skin contact electrode is applied to the skin of the subject.

2. The iontophoretic device of claim 1, wherein the first skin contact electrode and the second skin contact electrode are integrated in a patch.

3. The iontophoretic device (100) of claim 1, wherein the first ion-permeable barrier and the second ion-permeable barrier comprise respective salt bridges.

4. The iontophoretic device of claim 3, wherein each salt bridge comprises a gel including an isotonic NaCl concentration.

5. The iontophoretic device of claim 1, wherein the first ion-permeable barrier and the second ion-permeable barrier comprise respective ion-exchange membranes.

6. The iontophoretic device of claim 1 wherein:
the free sodium ion reservoir comprises an electrolyte solution including sodium ions; and
the free chloride ion reservoir comprises an electrolyte solution including chloride ions.

7. The iontophoretic device of claim 6, wherein the respective electrolyte solutions are buffered solutions.

8. The iontophoretic device of any of claim 1, wherein:
the sodium ion reservoir comprises a hydrogel including sodium ions; and
the chloride ion reservoir comprises a hydrogel including chloride ions.

9. The iontophoretic device of claim 1, further comprising an integrated DC voltage source having a first supply terminal conductively coupled to the first skin contact electrode and a second supply terminal conductively coupled to the second skin contact electrode.

10. An arrangement including the iontophoretic device of claim 1 and a DC supply source separate to the iontophoretic device for providing a direct voltage to the wearable iontophoretic device over a defined period of time, said DC supply source comprising a first supply terminal for conductively connecting to the first skin contact electrode and a second supply terminal for conductively connecting to the second skin contact electrode.

11. The arrangement of claim 10, wherein the DC supply source is adaptable to generate a cutaneous DC electric field in the range of 0.1-10 V/cm, or preferably in the range of 0.5-2 V/cm, such as about 1 V/cm.

12. A method of operating the iontophoretic device of claim 1, the method comprising:
bringing the device into contact with an area of skin such that the first skin contact electrode and the second skin contact electrode contact said area; and
generating a cutaneous DC electrical field across said area for a period of time by providing the first electrode and the second electrode with a potential difference for said period of time in order to induce asymmetric stem cell division in said area.

13. The method of claim 12, wherein said area comprises hair follicles, and said cutaneous DC electrical field is applied for a period of time sufficient to induce asymmetric stem cell division in said hair follicles.

14. The method of claim 12, wherein said period of time is at least 1 hour.
